(19) United States
(12) Patent Application Publication
(10) Pub. No.: US 2004/0247632 A1
(43) Pub. Date: Dec. 9, 2004

(54) Chitosan microparticles for the topical delivery of water insoluble active agents

(75) Inventor: Maurizio Cattaneo, Quincy, MA (US)

Correspondence Address: MAURIZIO CATTANEO, 196 SAMOSET AVE., QUINCY, MA, 02169, US

(73) Assignee: IVREA, INC.

(21) Appl. No.: 10/839,907

(22) Filed: May 6, 2004

Related U.S. Application Data
(63) Continuation of application No. 10/221,307, filed on Sep. 9, 2002.
Application No. 10/221,307 is a 371 of international application No. PCT/US00/35319, filed on Dec. 22, 2000.
(60) Provisional application No. 60/171,959, filed on Dec. 23, 1999.

(51) Int. Cl. A61K 31/203; A61K 7/00

(52) U.S. Cl. 424/401; 514/559

(57) ABSTRACT

In accordance with the present invention, there are provided high viscosity chitosan-based topical compositions for the delivery of water insoluble active agents (such as retinoic acid) in which the active agent is entrapped either dissolved or in the form of suspended particles in a suitable dispersing agent in a chitosan matrix under vigorous stirring conditions. The chitosan matrix containing the active ingredient is then mixed in the presence of an aqueous anionic polymeric solution at pH greater than 6.0 such as to cause precipitation under vigorous stirring conditions of the chitosan matrix in the form of microparticles.

### REFERENCES CITED

| | | | |
|---|---|---|---|
| U.S. Pat. App. No. | April, 2003 | Garces | 424/493 |
| 20030064106 | | | |
| U.S. Pat. App. No. | March, 2003 | Viladot | 424/490 |
| 20030044469 | | | |
| U.S. Pat. No. | June, 2001 | Grandmontagne | 428/402.2 |
| 6,242,099 | | | |
| U.S. Pat. No. | September, 1999 | Won | 424/501 |
| 5,955,109 | | | |

### STATEMENT REGARDING FED SPONSORED R&D

This work was sponsored in part by NIH Grant 2R44 CA086653

### FIELD OF INVENTION

The present invention relates to the topical delivery of water insoluble active agents (e.g. retinoids). The active agent is entrapped in a chitosan matrix, either dissolved or dispersed in a suitable dispersing agent under vigorous stirring conditions. The chitosan carrier is then precipitated using an anionic polymer at a pH greater than 6.0 to form microparticles containing the active agent.

### BACKGROUND OF THE INVENTION

Topical retinoids such as retinoic acid have been used to treat skin conditions such as acne, actinic keratosis, psoriasis, skin cancers and photodamage and chemoprevention of melanoma [Griffiths et al., N Eng J Med 329:530-534 (1993); Halpern et al., In: Advances in the biology and treatment of cutaneous melanoma, Boston, Mass., Nov. 6-7^{th}(1998); Kligman, J Am Acad Dermatol 39:S2-S7 (1998); Stam-Postuma, Melanoma Research 8:539-48 (1998); Varani et al. J Inv Dermatol 114:480-486 (2000)].

One side effect of topical retinoic acid for treating skin ailments is increased irritation. Topical tretinoin (ATRA) induces irritation in 90% of patients (Gilchrest, J Am Acad Dermatol 36:S27-S36 (1997)], and other side effects include patchy erythema, localized swelling, xerosis, and scaling. Irritation has been attributed, in part, by an overload of the tretinoin dependent pathways with non-physiological amounts of exogenous tretinoin in the skin. (Siegenthaler et al., In: Retinoids: From Basic Science To Clinical Applications, M. A. Livrea and G. Vidali (eds), Birkhauser Verlag, Basel, Switzerland, pp. 329-335, (1994)]. For example, compared to oral administration, topical delivery of retinoic acid increases the concentration of retinoic acid in the dermal compartment 10- to 100-fold (Lehman et al., J Invest Dermatol 91:56-61 (1988)]. This irritation may be the reason for discontinuation of treatment for approximately 50% of patients [(Stam-Postuma et al., Melanoma Research 8:539-48 (1998)]. This high incidence of irritation, leading to poor compliance, can preclude its use. Any means to reduce irritation is therefore seen as a very desirable attribute of any topical formulation. In the prior art, entrapment of retinoic acid in porous microspheres (Microsponge®) to slow down its release into the skin layers, resulted in a reduction of the level of irritation by controlling the release of the active into the skin [Won R, et al. U.S. Pat. No. 5,955,109 (1999)]. However, formulas containing this delivery system tend to deposit a fine dry residue on the skin surface which may not be cosmetically acceptable.

Microencapsulation has been used to deliver topical ingredients to reduce irritation and/or increase stability of the active ingredient. Various commercial microcapsules are available which differ in the type of polymers used to make the capsule wall such as Hallcrest Microcapsules (gelatin, gum arabic), Coletica Thalaspheres (collagen), Lipotec Millicapsules (alginic acid, agar agar), Induchem Unispheres (lactose, microcrystalline cellulose, hydroxypropyl-methylcellulose), Kobo Glycospheres (modified starch, fatty acid esters, phospholipids) and Softspheres (modified agar agar).

Chitosan is a natural, biodegradable cationic polysaccharide derived by deacetylating chitin, a natural material extracted from fungi, the exoskeletons of shellfish and from algae and has previously been described as a promoter of wound healing [Balassa, U.S. Pat. No. 3,632,754 (1972); Balassa, U.S. Pat. No. 3,911,116 (1975)]. Chitosan comprises a family of polymers with a high percentage of glucosamine (normally 70-99%) and N-acetylated glucosamine (1-30%) forming a linear saccharide chain of molecular weight from 10,000 up to about 1000,000 Dalton. Chitosan, through its cationic glucosamine groups, interacts with anionic proteins such as keratin in the skin conferring some bioadhesive characteristics. In addition, when not deacetylated, the acetamino groups of chitosan are an interesting target for hydrophobic interactions and contribute to some degree to its bioadhesive characteristics [(Muzzarelli et al., In: Chitin and Chitinases Jolles P and Muzzarelli RAA (eds), Birkhauser Verlag Publ., Basel, Switzerland, pp.251-264 (1999)].

In the prior art chitosan has been used mainly as a film forming and a moisturizing agent in skin and hair care preparations. Company literature indicates that chitosan being a polycationic polymer will form insoluble precipitates in the presence of anionic polymers and at a pH greater than 6 (refer to Cognis Company Literature on Hydagen® CMF and Amerchol Company literature on Kytamer™ PC). It is also our experience that chitosan is incompatible with anionic polymers and at pH greater than 6. The chitosan precipitate is in the form of a gel complex which may be end up as cosmetically unacceptable relatively large particulates in the final topical formula. However, this tendency of chitosan to precipitate in the presence of anionic substances, and/or higher pH values has been exploited in the prior art to form microencapsulated active agents.

In Grandmontagne et al. [U.S. Pat. No. 6,242,099 (2001)] microcapsules made of chitin or chitin derivative enveloping an hydrophobic substance were made using an anionic surfactant and chitosan. The anionic surfactant plays the dual role of emulsifying an hydrophobic substance as well as precipitating the chitosan polymer. The chitosan was further processed by crosslinking or formed into chitin by acetylation. In this invention the formation of microcapsules requires the use of anionic surfactants which may cause adverse skin reactions such as erythema and edema. In the use of surfactants to precipitate chitosan, reference is also made to German patent applications DE 19712978 A1 and DE 19756452 A1 which describe microspheres made by mixing chitosans or chitosan derivatives with oil bodies and precipitating these mixtures into alkaline surfactant solutions.

Garces J et al., describe microcapsules of 0.1 mm to 5 mm in diameter [U.S. patent application No. 20030064106] made by encapsulating an emulsion of the active ingredient with an anionic polymer followed by chitosan. These microcapsules are significantly larger than the object of this invention, and they were obtained by a different method which included emulsifiers to form the initial emulsion and solubilize the active ingredient.

All of the above encapsulation methods use surfactancts and/or emulsifiers as a critical step in the making of the chitosan-based microparticulates. These surfactants, especially the anionic surfactants can contribute to increased skin irritation and other adverse skin reactions. In addition, some of these microparticulates leave a cosmetically unacceptable residue after topical application. The object of this invention was then to overcome these disadvantages.

### BRIEF DESCRIPTION OF THE INVENTION

Thus it is the object of this invention to topically deliver active agents (e.g. retinoic acid, retinol, calcipotriene) in unmodified form in a composition that minimizes adverse skin reactions such as erythema or edema due in part to the presence of added emulsifiers and surfactants, as currently employed in topical drug delivery.

It is a further object of this invention to topically deliver active agents suspended in suitable dispersing agent contained in microparticles composed of high viscosity chitosan.

It is yet another object of this invention to topically deliver active agents in a composition of microparticles which does not leave residual microparticles on the topical surface.

These and other objects of the invention will become apparent upon review of the specification and claims.

In accordance with the present invention, I have discovered that water insoluble active agents can be delivered in the form of microparticles that are suitable for topical administration. This mode of delivery obviates the need for administration of insoluble active agents (e.g. retinoic acid) in an emulsion containing for example ethanol and polyethoxylated castor oil, diluted in a poly(acrylic acid) gel (See for example Technical Bulletin ME 142e, Tretinoin for the Pharmaceutical Industry, October 1998, BASF Corporation). A disadvantage of such known compositions is their propensity to cause adverse skin reactions such as erythema and swelling.

The topical delivery of water insoluble active agents in the form of a chitosan particulate suspension allows greater stability of the active ingredient and increased penetration of the microparticles in the stratum corneum, which is the outer layer of the skin.

In accordance with another embodiment of the present invention, I have developed compositions useful for topical delivery of insoluble and slightly water soluble active agents. Invention compositions comprise water insoluble or slightly water soluble active agents (as a solid or a liquid) contained in a polymeric matrix. The polymeric matrix consists of a high viscosity chitosan biopolymer and a suitable dispersing agent which is precipitated under vigorous stirring conditions in the presence of anionic polymers and/or pH values greater than 6.0 to form microparticles of size less than 100 micrometers.

### DESCRIPTION OF THE INVENTION

It was found, unexpectedly, that if a high viscosity chitosan is first mixed in the presence of a water insoluble active ingredient dispersed in a suitable solvent to form a matrix, this matrix can then be precipitated under vigorous stirring conditions in the presence of anionic polymers and at higher pH values to form micron size particles which penetrate the stratum corneum or outer skin layer: This preparation of chitosan-based microparticles avoids the use of surfactants or emulsifiers which can cause skin irritation or other adverse reactions. The method of the present invention provides a system for incorporating water insoluble or slightly soluble active agents, e.g., pharmaceuticals, such as retinoids, into polymeric carriers to provide advantages, such as preferable tissue distribution of the drug, prolonged half life, controlled drug release and reduction of drug toxicity. More particularly, the present invention relates to the use of chitosan microparticles for the topical delivery of water insoluble active agents, e.g., retinoids, where the sustained release of the drug is obtained by precipitating the chitosan/active agent matrix in the presence of anionic polymers at pH conditions greater than 6.0 under vigorous stirring conditions. In addition, the chitosan microparticles disclosed in the present invention are able to act as delivery vehicles without leaving polymeric residues on the skin as evidenced in the prior art. The absence of residues may be due to the bioadhesiveness of chitosan to the skin surface as mentioned earlier which allows for greater penetration into the stratum corneum or the outer layer of the skin. Consistent with the ability of delivery vehicles to reduce skin irritation, the chitosan-based microparticles show statistically lower levels of both erythema and edema in animal studies as shown in the examples.

As used herein, the term “active agent” refers to any substance that when introduced into the body has an effect on either the appearance of tissue to which it is applied, or alters the way the body functions.

The term “water insoluble” refers to any active agent insoluble in water or slightly water soluble.

The term “pharmaceutical active” refers to a drug, i.e., a substance which when applied to, or introduced into the body, alters in some way body functions, e.g., altering cell processes. Examples of water insoluble or slightly water soluble pharmaceutical actives include, but are not limited to, agents that are used for the treatment of skin diseases, e.g., retinoids, corticosteroids, non-steroidal anti-inflammatory drugs (NSAIDS), hormones, anti-fungal agents, anti-septic agents, local anaesthetics, keratolytic agents, skin bleaching agents, etc. Examples of these actives include, but are not limited to compounds such as the following: retinoids such as retinoic acid (both cis and trans) and tazarotene, anti-inflammatory analgesics such as salicylic acid, salicylate esters, acetylsalicylic acid, diflunisal, phenylbutazone, oxyphenbutazone, slightly water soluble steroids (e.g. estrogen), ibuprofen, ketoprofen, naproxen, mefenamic acid, floctafenine, tolmetin, zomepirac, diclofenac, piroxicam, and the like. Local anaesthetics such as cocaine, benzocaine, tetracaine, lidocaine, bupivacaine, their hydrochloride salts, and the like. Antibiotic agents such as penicillins, cephalosporins, cyclosporin, vancomycin, bacitracin, cycloserine, polymyxins; colistin, nystatin, amphotericin B, mupirocim, tetracyclines, chloramphenicol, erythromycin, neomycin, streptomycin, kanamycin, gentamicin, tobramycin, amikacin, netilmicin, spectinomycin, clindamycin, rifampin, nalidixic acid, flucytosine, griseofulvin, and the like. Sulfanilamide antibacterial agents such as sulfanilamide, sulfacetamide, sulfadiazine, sulfisoxazole, sulfamethoxazole, trimethoprim, pyrimethamine, and the like. Antiviral agents such as vidarabine, acyclovir, ribavirin, amantadine hydrochloride, rimantadine, idoxyuridine, interferons, and the like. Antiseptic agents such as acridine dyes, bronopol, chlorhexidine, phenols, hexachlorophene, organic mercurials, organic peroxides, i.e., benzoyl peroxide, quaternary ammonium compounds, and the like. Anti-inflammatory corticosteroids such as progesterone, hydrocortisone, prednisone, fludrocortisone, triamcinolone, dexamethasone, betamethasone, fluocinolone, and the like. Anti-fungal agents such as miconazole, tolnaftate, undecylic acid, and other heterocyclic compounds including morpholine, imidazoles and derivatives thereof. Autacoids such as prostaglandins, prostacyclin, thromboxanes, leukotrienes, angiotensins (captopril), as well as other pharmaceutically active peptides such as serotonin, endorphins, vasopressin, oxytocin, and the like. Depigmenting agents such as hydroquinone and kojic acid. Keratolytic agents such as benzoyl peroxide, salicylic acid, trichloroacetic acid, and piroctone, and wart treatment compounds such as salicyclic acid, trichloroacetic acid and lactic acid, singularly or in combination with anti-viral agents. Anti-alopecia agents such as niacin, nicotinate esters and salts, and minoxidil.

The term “therapeutic active” as used herein, refers to insoluble or slightly water soluble substances which either alters processes within the body, or alters the cosmetic appearance of the tissue of interest, e.g., skin, but is not technically considered a drug. Examples of therapeutic actives include, but are not limited to, vitamins and vitamin derivatives such as Vitamin A, retinol, alpha-tocopherol (Vitamin E), 7-dehydrocholesterol (Vitamin D), Vitamin K, alpha-lipoic acid, lipid soluble anti-oxidant and the like.

The term “chromogenic active”, as used herein refers to water insoluble or slightly water soluble sunscreens. Examples of sunscreens are octylmethoxycinnamate and related esters, octyl salicylate and esters, para-aminobenzoic acid and esters, benzophenones such as 2-hydroxy-4-methoxybenzophenone, benzyldiphenyl acrylates, anthranilates, triazines, benzylidenecamphor and derivatives.

In certain embodiments, the compositions' contains more than one active agent, thus the compositions comprises at least one additional active agent, which can be either a pharmaceutical active or a therapeutic active. For example, in a preferred embodiment, the compositions includes a retinoid as a pharmaceutical active and vitamin E as a therapeutic active.

The amount of water insoluble active employed will be that amount necessary to deliver a pharmaceutically or therapeutically effective amount to achieve the desired result at the site of application. In practice, this will vary depending upon the particular medicament, severity of the condition as well as other factors. In general, the concentration of the actives in the final formula can vary from as little as 0.0001 up to 20 percent or higher, by weight of the final formula. For retinoids, a preferred dose is between 0.01%-1% for retinol and between 0.01% -0.1% for all-trans-retinoic acid.

The invention will be discussed in relation to retinoids. However, it is to be understood that any active agent that can be used in a topical delivery system can be used in the compositions and methods of the present invention. Preferably the active agent is a water insoluble substance. For example, preferred agents include retinoids, e.g., retinoic acid and retinol (Vitamin A), calcipotriene, and other active agents which are known to cause irritation of the skin.

The term “topical” as used herein is known in that art and includes the application of the compounds of the present invention to skin surfaces, including mucosal membranes of the upper respiratory, digestive and gastrointestinal tract.

The term “biopolymer” as used herein includes a component of the delivery system that assists in the release of the active agent that is being delivered. Preferred biopolymers comprise a high viscosity chitosan having a molecular weight of at least about 100,000 Dalton, more preferably at least about 250,000 Dalton and most preferably at least about 300,000 Dalton. In other preferred embodiments the chitosan has a concentration of at least about 2 weight %. In an especially preferred embodiment, the biopolymer comprises a high viscosity chitosan having a molecular weight of at least about 300,000 Dalton and at a concentration of at least 2 weight %.

The term “high viscosity” chitosan refers to a chitosan biopolymer having an apparent viscosity of at least about 100 cps for 1% solutions in 1% acetic acid as measured using a Brookfield LVT viscometer at 25° C. with appropriate spindle at 30 rpm. The viscosity of the chitosan solution can readily be determined by one of ordinary skill in the art, e.g., by the methods described in Li et al., Rheological Properties of aqueous suspensions of chitin crystallites. J Colloid Interface Sc 183:365-373, 1996. In addition, viscosity can be estimated according to Philipof's equation: V=(1+KC)⁸, where V is the viscosity in cps, K is a constant, C is the concentration expressed as a fraction (Form No. 198-1029-997GW, Dow Chemical Company). In certain embodiments, the high viscosity chitosan preferably has a viscosity greater than at least 100 cps, and more preferably greater than at least 500 cps.

The term “dispersing agent” as used herein comprises any suitable solvent that will solubilize or suspend the water insoluble or slightly water soluble active agent but does not chemically react with either chitosan or the active substance. Examples include soybean oil, dibutyl hexanedioate, cocoglycerides, aliphatic or aromatic esters having 2-30 carbon atoms (e.g. cococaprylate/caprate), coconut oil, olive oil, safflower oil, cotton seed oil, alkyl, aryl, or cyclic ethers having 2-30 carbon atoms, cycloaliphatic or aromatic hydrocarbons having 4-30 carbon atoms, alkyl or aryl halides having 1-30 carbon atoms.

The term “anionic polymer” refers to negatively charged polymers which can form a complex with chitosan such as poly(acrylic acid) and derivatives, xanthan gum, sodium alginate, gum arabic, carboxy methylcellulose, hydroxypropyl methylcellulose, ethylcellulose, carrageenan, polyvinyl alcohol, sulfated glycosaminoglycans such as chondroitin sulfate and dermatan sulfate.

The compositions and methods of the present invention rely on the discovery that the active ingredient can be entrapped in a high viscosity chitosan. The desired viscosity of the chitosans can be achieved by manipulating the concentration, i.e., percentage, of different molecular weight chitosans. To produce the microparticles according to the invention, a matrix consisting of a viscous aqueous solution of chitosan, the oil component and the water insoluble active agent is formed by vigorous stirring in a first step. The molecular weight of chitosan is preferably above 100,000 Dalton and at a concentration above 2 wt %. The chitosan matrix is then precipitated by adding an anionic polymer solution at a pH greater than 6 which results in the formation of microparticles under vigorous stirring.

The invention will now be described in greater detail by reference to the following non-limiting examples:

### EXAMPLE 1

Preparation of Retinoic Acid Particles

Water-insoluble all-trans retinoic acid (ATRA) in the form of solid particles (2 wt %) was incorporated into high viscosity chitosan solutions [3 wt % solutions of Protasan UP B 80/500 (FMC Biopolymers Inc.; 755 cps apparent viscosity) in 2.1 wt % glycolic acid and 0.03 wt % sodium hydroxide] in the presence of soybean oil (17 wt % ) by vigorous mixing to form a matrix. The viscosity of the matrix was initially 215,000 cps as measured on a Brookfield LVT viscometer at 25° C. with appropriate spindle at 1.5 rpm. The emulsion was then mixed with a poly(acrylic acid) solution (0.5 wt %) at pH 6.3 and homogenized to make a gel containing retinoic acid microparticles of size below 10 microns.

### EXAMPLE 2

Stability of Retinoic Acid Particles

The concentration of retinoic acid in the final gel formulation was measured by HPLC. Fifty microliters of retinoic acid microparticulate gel extracted by Fifty microliters of the topical preparation containing retinoic acid was shaken for 20 minutes in the presence of 5 milliliters of acetonitrile then centrifuged at 4000 rpm for 5 minutes. A 20 microliter aliquot of the supernatant was then injected onto a Zorbax SB-C18 column (4.6 mm×75 mm, 3.5 micron) equipped with a Zorbax SB-C18 Guard cartridge (4.6×12.5 mm) and operated with aq. 70% acetonitrile containing 5% acetic acid and 0.02% triethanolamine as mobile phase (1 ml/min) and detection at 340 nm. The calibration was linear from 50 to 5,000 ng/ml.

The stability of the retinoic acid was determined over a 3 month period. The retinoic acid was highly stable in the chitosan microparticulates. The initial retinoic acid concentration was determined as 0.052% at time 0 and 0.05% at 3 months.

### EXAMPLE 3

Preclinical Study Involving Gel Formulation Containing Retinoic Acid Particles

A 3-months preclinical study was undertaken in both mice and rabbits to determine the severity of skin reactions after application of the retinoic acid gel as described above using the Draize test. The animals (40 New Zealand White Rabbits and 140 CD-1 mice) were divided into 5 groups as shown in Table 1, 2 and 3.

The test compound was formulated to include a concentration of 0.05 wt % of retinoic acid in microparticulate form as illustrated in Example 1 and applied at 100 times and 500 times the human dose (Groups 3 and 4). The vehicle gel and the vehicle gel containing the chitosan microparticles without retinoic acid (Groups 1 and 2) acted as negative controls whereas a commercial 0.05% cream (Renova 0.05% retinoic acid) in a standard emulsion formula at 500 the human dose (Group 5) acted as positive control. As shown in Table 1 in the rabbit study, it was soon apparent that the positive control was too irritating for the animals and three steps were taken to manage the toxicity of the positive control group: (1) the positive control dose was scaled back to 100 times the human dose from 500 the human dose after 10 days of application; (2) a second site of application of the positive control was required while waiting for the first site to heal (about 2 weeks later) and (3) the animals which displayed the greatest discomfort were given an intramuscular injection of buprenorphine (2 out of 8 animals). As shown in Table 1, the microparticulate delivery system alone did not cause erythema or edema and treatment groups 3 and 4 showed a statistically significant lower irritation and edema level compared to group 5.

TABLE 1
Rabbit study - Average Erythema and Edema Scores
at 10 days post treatment

| | Average | Average | |
|---|---|---|---|
| | Erythema | Edema | |
| Group Number | (n = 8) | (n = 8) | Comment |
| 1. Vehicle | 0.125 | 0 | No erythema/edema |
| 2. Vehicle + Microparticles (no ATRA) | 0 | 0 | No erythema/edema |
| 3. Vehicle + ATRA Microparticles | 0.125 | 0 | No erythema/edema |
| (100 human dose) | | | |
| 4. Vehicle + ATRA Microparticles | 0.875 | 0.25 | Very Slight erythema; |
| (500 human dose) | | | no edema |
| 5. Renova ® (500 human dose) | 2.125 | 1.875 | Well defined erythema; |
| | | | slight edema |

After treating the second application site with Renova® at the lower dose (100 times the human dose) for an additional 14 days the level of erythema and edema significantly exceeded the test compound at 100 and the 500 times the human dose (Table 2).

TABLE 2
Rabbit study - average erythema and edema
scores at 24 days post-treatment

| | Average | Average | |
|---|---|---|---|
| | Erythema | Edema | |
| Group Number | (n = 8) | (n = 8) | Comment |
| 1. Vehicle | 0.125 | 0 | No erythema/edema |
| 2. Vehicle + Microparticles | 0 | 0 | No erythema/edema |
| 3. Vehicle + ATRA | 0 | 0 | No erythema/edema |
| Microparticles | | | |
| (100 times the human dose) | | | |
| 4. Vehicle + ATRA | 1.75 | 0.75 | Slight erythema; |
| Microparticles | | | very slight edema |
| (500 times the human dose) | | | |
| 5. Renova ® | 2.125 | 1.5 | Well defined |
| (500 times the human | | | erythema; slight |
| dose)* | | | edema |

*Applied on second site starting at day 10

In the mice study, at 10 days post treatment the positive control group (Group 5—Renova®) receiving 100 times the human dose had significantly more erythema than Group 4 treated with 500 times the human dose of test compound (Table 3):

TABLE 3
Mice Study - Average Erythema and Edema
Scores at 10 days post-treatment

| | Average | Average | |
|---|---|---|---|
| | Erythema | Edema | |
| Group Number | (n = 28) | (n = 28) | Comment |
| 1. Vehicle | 0 | 0 | No erythema/edema |
| 2. Vehicle + Microparticles | 0.07 | 0 | No erythema/edema |
| 3. Vehicle + ATRA Microparticles | 0.57 | 0.25 | Very slight erythema; no edema |
| (100 times the human dose) | | | |
| 4. Vehicle + ATRA Microparticles | 1.0 | 0.46 | Very slight erythema; no edema |
| (500 times the human dose) | | | |
| 5. Renova ® | 1.67 | 1.21 | Well defined erythema; |
| (500 times the human dose)* | | | slight edema |

The preclinical results indicate that the test compound is significantly less irritating than a commercial retinoic acid preparation (Renova® 0.05%) in both rabbit and mice studies.

These results strongly suggest the potential for increasing patient compliance in patients undergoing retinoic acid therapy for skin diseases such as acne, photodamage and prevention of melanoma.

### EXAMPLE 4

Preparation of Alpha-Lipoic Acid Particles.

The slightly water soluble substance alpha lipoic acid (1.5 wt %) was mixed with an aqueous high viscosity chitosan solution (8.3 wt %, 40,000 cps) and soybean oil (0.8 wt %) under vigorous stirring. The pH of the emulsion was then raised to 6.3 using triethanolamine under vigorous stirring conditions to precipitate the chitosan matrix (no anionic polymer was used in this procedure). The microparticle size was 5 microns. The emulsion was then passed through a Microfluidizer® to obtain particle sizes approximately 500 nm in diameter after 5 passes through the Microfluidizer® filters. The microfluidizer pushes the emulsion through very fine pore filters at high pressure (greater than 1000 psi) which causes a reduction in particle size.

### EXAMPLE 5

Preparation of Octylmethoxycinnamate Sunscreen Particles.

The slightly water soluble sunscreen octylmethoxycinnamate (7.5 wt %, 40,000 cps) was first mixed with an aqueous high viscosity chitosan solution (10 wt %) to form an oil in water (O/W) emulsion. The emulsion was then mixed with an aqueous xanthan gum solution (40 wt %) to further increase the viscosity of the sunscreen/chitosan (O/W) emulsion. In a separate container microfine zinc oxide (9 wt %) was mixed with an oily solution containing cocoglycerides (12 wt %), lauryl glucoside (3 wt %), polyglyceryl-2-dipolyhydroxystereate (1 wt %) and sodium cetearyl sulfate (1 wt %) heated at 70° C. The sunscreen/chitosan O/W emulsion and the oily solution containing microfine zinc oxide were then mixed together using a high speed mixer. The final pH of the sunscreen was 7.0 which caused precipitation of the chitosan matrix in the form of microparticles containing the sunscreen agent. The mixture was then cooled below 40° C. before adding preservative.

### EXAMPLE 1

Preparation of Retinoic Acid Particles

Water-insoluble all-trans retinoic acid (ATRA) in the form of solid particles (2 wt %) was incorporated into high viscosity chitosan solutions [3 wt % solutions of Protasan UP B 80/500 (FMC Biopolymers Inc.; 755 cps apparent viscosity) in 2.1 wt % glycolic acid and 0.03 wt % sodium hydroxide] in the presence of soybean oil (17 wt % ) by vigorous mixing to form a matrix. The viscosity of the matrix was initially 215,000 cps as measured on a Brookfield LVT viscometer at 25° C. with appropriate spindle at 1.5 rpm. The emulsion was then mixed with a poly(acrylic acid) solution (0.5 wt %) at pH 6.3 and homogenized to make a gel containing retinoic acid microparticles of size below 10 microns.

### EXAMPLE 2

Stability of Retinoic Acid Particles

The concentration of retinoic acid in the final gel formulation was measured by HPLC. Fifty microliters of the topical preparation containing retinoic acid was shaken for 20 minutes in the presence of 5 milliliters of acetonitrile then centrifuged at 4000 rpm for 5 minutes. A 20 microliter aliquot of the supernatant was then injected onto a Zorbax SB-C18 column (4.6 mm×75 mm, 3.5 micron) equipped with a Zorbax SB-C18 Guard cartridge (4.6×12.5 mm) and operated with aq. 70% acetonitrile containing 5% acetic acid and 0.02% triethanolamine as mobile phase (1 ml/min) and detection at 340 mn. The calibration was linear from 50 to 5,000 ng/ml.

The stability of the retinoic acid was determined over a 3 month period. The retinoic acid was highly stable in the chitosan microparticulates. The initial retinoic acid concentration was determined as 0.052 % at time 0 and 0.05% at 3 months.

### EXAMPLE 3

Preclinical Study Involving Gel Formulation Containing Retinoic Acid Particles

A 3-months preclinical study was undertaken in both mice and rabbits to determine the severity of skin reactions after application of the retinoic acid gel as described above After treating the second application site with Renova® at the lower dose (100 times the human dose) for an additional 14 days the level of erythema and edema significantly exceeded the test compound at 100 and the 500 times the human dose (Table 2).

TABLE 2
Rabbit study - average erythema and edema
scores at 24 days post-treatment

| | Average | Average | |
|---|---|---|---|
| | Erythema | Edema | |
| Group Number | (n = 8) | (n = 8) | Comment |
| 1. Vehicle | 0.125 | 0 | No erythema/edema |
| 2. Vehicle + Microparticles | 0 | 0 | No erythema/edema |
| 3. Vehicle + ATRA | 0 | 0 | No erythema/edema |
| Microparticles | | | |
| (100 times the human dose) | | | |
| 4. Vehicle + ATRA | 1.75 | 0.75 | Slight erythema; |
| Microparticles | | | very slight edema |
| (500 times the human dose) | | | |
| 5. Renova ® | 2.125 | 1.5 | Well defined |
| (100 times the human | | | erythema; slight |
| dose)* | | | edema |

*Applied on second site starting at day 10

In the mice study, at 10 days post treatment the positive control group (Group 5—Renova®) receiving 100 times the human dose had significantly more erythema than Group 4 treated with 500 times the human dose of test compound (Table 3):

TABLE 3
Mice Study - Average Erythema and Edema
Scores at 10 days post-treatment

| | Average | Average | |
|---|---|---|---|
| | Erythema | Edema | |
| Group Number | (n = 28) | (n = 28) | Comment |
| 1. Vehicle | 0 | 0 | No erythema/edema |
| 2. Vehicle + Microparticles | 0.07 | 0 | No erythema/edema |
| 3. Vehicle + ATRA Microparticles | 0.57 | 0.25 | Very slight erythema; no edema |
| (100 times the human dose) | | | |
| 4. Vehicle + ATRA Microparticles | 1.0 | 0.46 | Very slight erythema; no edema |
| (500 times the human dose) | | | |
| 5. Renova ® | 1.67 | 1.21 | Well defined erythema; |
| (100 times the human dose)* | | | slight edema |

The preclinical results indicate that the test compound is significantly less irritating than a commercial retinoic acid preparation (Renova® 0.05%) in both rabbit and mice studies.

What is claimed is:

1. A method for the preparation of topical compositions containing water insoluble or slightly water soluble active agents, said method comprising forming a homogeneous matrix consisting of an aqueous solution of a high viscosity chitosan biopolymer and the active agent, either soluble or suspended in a suitable dispersing agent, and precipitating the matrix by using an anionic polymer solution at pH greater than 6.0, under vigorous stirring conditions to produce microparticles with mean diameter of no greater than about 100 microns.

2. Topical compositions, according to claim 1, containing an effective quantity of microparticles which are obtained under vigorous stirring conditions by (a) forming a matrix consisting of aqueous solutions of a high viscosity chitosan polymer with an active agent, either soluble or dispersed in a suitable dispersing agent and (b) precipitating the matrix by increasing the pH to greater than 6.0.

3. Preparations as claimed in 1, characterized in that the microparticles have a retinoic acid content of 0.001 to 5% by weight.

4. The method according to claim 1, wherein chitosan has a molecular weight of at least about 100,000 Dalton and a concentration of at least about 2 weight %.

5. The method according to claim 1, wherein the microparticles are present in quantities of 0.1 to 10% by weight, based on the final preparation.

6. The method according to claim 1 wherein said dispersing agent is selected from soybean oil, mineral oil, olive oil, almond oil, coconut oil, safflower oil, cotton seed oil, dibutyl hexanedioate, cocoglycerides, cococaprylate/caprate, alkyl, aryl, or cyclic ethers having 2-30 carbon atoms, cycloaliphatic or aromatic hydrocarbons having 4-30 carbon atoms, alkyl or aryl halides having 1-30 carbon atoms, ketones having 3-30 carbon atoms and volatile dispersing agents such as dichloromethane, ethyl acetate, benzene and the like.

7. The method according to claim 1 wherein said anionic polymer is selected from anionic polysaccharides, poly(acrylic acid) and derivatives, sodium alginate, polyvinyl alcohol, sulfated glycosaminoglycans including dermatan sulfate and chondroitin sulfate and anionic chitosan derivatives such as carboxylated and succinylated chitosan.

8. The method according to claim 1, wherein the slightly water soluble active is a pharmaceutical active or a therapeutic active or a chromogenic active agent.

9. The method according to claim 8, wherein the pharmaceutical active is used for the treatment of skin diseases.

10. The method according to claim 9, wherein the pharmaceutical active is selected from retinoids, steroids, calcipotriene, lidocaine, benzocaine, diazepam, hydroquinone, ketoconazole, miconazole, spectazole, econazole, aspirin, ibuprofen, cyclosporines, lipid soluble hormones, anti-fungal agents, anti-septic agents, local anaesthetics and kerolytic agents.

11. The method according to claim 10, wherein the pharmaceutical active is retinoic acid.

12. The method according to claim 8, wherein the therapeutic agent is selected from lipid soluble vitamin A, D, E and K and derivatives, alpha lipoic acid, lipid soluble anti-oxidants, aromatic oils, orange oil, seabuckthorn oil, fragrances and the like.

13. The method according to claim 12, wherein the therapeutic agent is retinol.

14. The method according to claim 8, wherein the chromogenic agent is a water insoluble sunscreen.

15. The method according to claim 8, wherein the chromogenic agent is octylmethoxycinnamate.

16. Compositions according to claim 8, in the form of a gel, a cream or a lotion.
